(19) United States
(12) Patent Application Publication
(10) Pub. No.: US 2007/0167422 A1
(43) Pub. Date: Jul. 19, 2007

(54) Pharmaceutical compositions comprising 17-allylamino-17-demethoxygeldanamycin

(76) Inventors: Kwok S. Yu, San Mateo, CA (US); Ziyang Zhong, Union City, CA (US)

Correspondence Address: KOSAN BIOSCIENCES, INC, 3832 BAY CENTER PLACE, HAYWARD, CA, 94588, US

(21) Appl. No.: 11/637,566

(22) Filed: Dec. 11, 2006

Related U.S. Application Data
(60) Provisional application No. 60/760,142, filed on Jan. 18, 2006.

(51) Int. Cl. A61K 31/395; A61K 31/355

(52) U.S. Cl. 514/183; 514/458

(57) ABSTRACT

A pharmaceutical formulation comprising (a) 17-allylamino-17-demethoxy-geldanamycin; (b) an ester of d-α-tocopheryl succinate and polyethylene glycol; and (c) a pharmaceutically acceptable, water-miscible organic solvent.

### CROSS-REFERENCE TO RELATED APPLICATIONS

This application claims the benefit under 35 U.S.C. §119(e) of U.S. Provisional Application No. 60/760,142, filed Jan. 18, 2006, the disclosure of which is incorporated herein by reference.

### BACKGROUND OF THE INVENTION

1 . Field of the Invention

This invention relates to pharmaceutical formulations containing 17-allylamino-17-demethoxygeldanamycin (“17-AAG”) and methods for their preparation and use.

2. Description of Related Art

Geldanamycin belongs to the ansamycin family of natural products, whose members are characterized by a benzenoid nucleus (typically a benzoquinone or hydroquinone nucleus) connected at two meta positions to form a macrolactam. Besides geldanamycin, the ansamycins include the macbecins, the herbimycins, the TAN-420s, and reblastatin.

Geldanamycin and its derivatives are the most extensively studied of the ansamycins. Although geldanamycin originally was identified as a result of screening for antibiotic activity, current interest arises from its potential as an anticancer agent. It is an inhibitor of heat shock protein-90 (“Hsp90”), a so-called “chaperone protein” involved in the folding and activation of numerous proteins (“client proteins”), including key proteins involved in signal transduction, cell cycle control and transcriptional regulation. The binding of an inhibitor to Hsp90 disrupts interactions with a client protein, preventing the client protein from being folded correctly and rendering it susceptible to proteasome-mediated destruction. Among the Hsp90 client proteins are many mutated or overexpressed proteins implicated in cancer, such as mutant p53, Bcr-Abl kinase, Raf-1 kinase, Akt kinase, Npm-Alk kinase, Cdk4, Cdk6, Wee1, HER2/Neu (ErbB2), and hypoxia inducible factor-1α (HIF-1α). The possibility that multiple oncogenic client proteins can be simultaneously targeted has generated considerable interest in the development of Hsp90 inhibitors as anti-cancer drugs. See, e.g., Xiao et al., Mini-Reviews Med. Chem. 2006, 6 (10), 1137-1143.

Geldanamycin has been considered for development as an Hsp90-inhibiting anti-cancer drug, but its hepatotoxicity and poor bioavailability have led to its discontinuation as a clinical candidate. Nevertheless, interest persists in the development of ansamycins having Hsp90 inhibitory activity, but with a more pharmaceutically acceptable spectrum of properties. Position 17 of geldanamycin has been an attractive focal point, chemically speaking, for the synthesis of geldanamycin derivatives because its methoxy group is readily displaced by a nucleophile, providing a convenient synthetic pathway to the 17-substituted-17-demethoxygeldanamycins. Structure-activity relationship (“SAR”) studies have shown that chemically and sterically diverse 17-substituents can be introduced without destroying antitumor activity. See, e.g., Sasaki et al., U.S. Pat. No. 4,261,989 (1981) (hereinafter “Sasaki”); Schnur et al., U.S. Pat. No. 5,932,566 (1999); Schnur et al., J. Med. Chem. 1995, 38 (19), 3806-3812; Schnur et al., J. Med. Chem. 1995, 38 (19), 3813-3820; and Santi et al., U.S. Pat. No. 6,872,715 B2 (2005); the disclosures of which are incorporated by reference. The SAR inferences are supported by the X-ray crystal co-structure of the complex between Hsp90 and a geldanamycin derivative, showing that the 17-substituent juts out from the binding pocket and into the solvent (Jez et al., Chemistry & Biology 2003, 10, 361-368). The best-known 17-substituted geldanamycin derivatives are 17-allylamino-17-demethoxygeldanamycin (“17-AAG”, Sasaki, cited supra) and 17-(2-dimethylaminoethyl)amino-17-demethoxygeldanamycin (“17-DMAG”, Snader et al., U.S. Pat. No. 6,890,917 B2 (2005)), both of which are currently undergoing clinical trials.

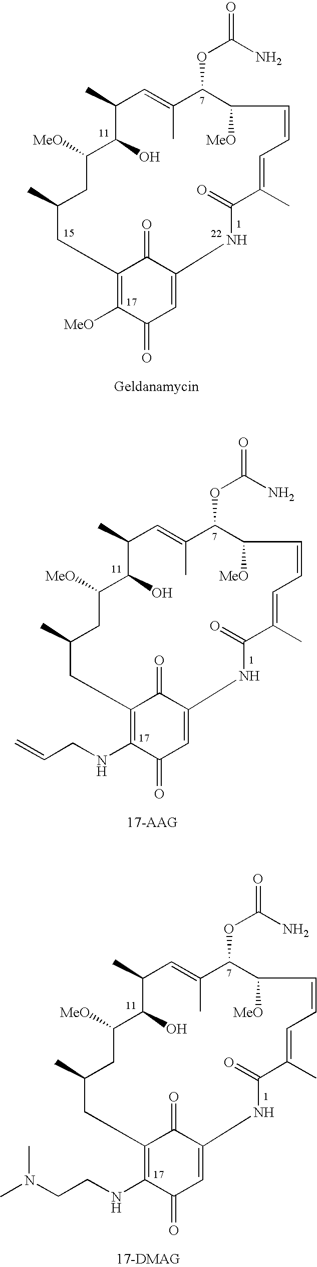

A limitation in the preparation of pharmaceutical formulations containing geldanamycin compounds such as geldanamycin itself and 17-AAG, especially for parenteral administration, is their very poor water solubility. (17-DMAG, having an alkyl amino group, is more soluble.) Addressing this issue, various disclosures have been made regarding formulations comprising 17-AAG:
(a) Tabibi et al., U.S. Pat. No. 6,682,758 B1 (2004) disclosed a formulation for a water insoluble drug such as 17-AAG comprising (a) the drug, (b) a water-miscible organic solvent for the drug, (c) a surfactant, and (d) water. The water miscible solvent can be dimethylsulfoxide (DMSO), dimethylformamide, ethanol, glycerin, propylene glycol, or polyethylene glycol. The surfactant preferably is a phospholipid (especially egg phospholipid).
(b) Ulm et al., WO 03/086381 (2003), which discloses a method for preparing pharmaceutical formulations for ansamycins by (a) providing the ansamycin dissolved in ethanol; (b) mixing the product of step (a) with a medium chain triglyceride to form a first mixture; (c) substantially removing the ethanol from the first mixture; (d) combining the product of step (c) with an emulsifying agent and a stabilizer to form a second mixture; and (e) emulsifying the second mixture. The emulsified second mixture optionally can be lyophilized and then re-hydrated. In a specific combination, the medium chain triglyceride comprises caprylic and/or caproic acid, the emulsifying agent comprises phosphatidylcholine, and stabilizer comprises sucrose.
(c) Ulm et al., WO 2004/082676 A1 (2004) discloses a pharmaceutical composition comprising an Hsp90 inhibitor such as 17-AAG, an emulsifying agent, and an oil comprising both medium and long chain triglycerides.
(d) Zhong et al., US 2005/0256097 A1 (2005) discloses a formulation of 17-AAG in a vehicle comprising (i) a first component that is ethanol; (ii) a second component that is a polyethoxylated castor oil (e.g., Cremophor™); and (iii) optionally a third component that is selected from the group consisting of propylene glycol, PEG 300, PEG 400, glycerol, and combinations thereof.
(e) Tao et al., Am. Assoc. Cancer Res., 96th Annual Meeting (Apr. 16-20, 2005), abstract no. 1435, discloses a nanoparticle albumin bound 17-AAG formulation asserted to be suitable for intravenous administration.
(f) Isaacs et al., WO 2006/094029 A2 (2006), discloses a pharmaceutical formulation comprising 17-AAG dissolved in a vehicle comprising an aprotic, polar solvent and an aqueous mixture of long chain triglycerides.
(f) Mansfield et al., US 2006/0067953 A1 (2006), discloses a pharmaceutical formulation for oral administration, comprising an ansamycin and one or more pharmaceutically acceptable solubilizers, with the proviso that when the solubilizer is a phospholipid, it is present in a concentration of at least 5% w/w of the formulation. Other solubilizers disclosed include polyethylene glycols of various molecular weights, ethanol, sodium lauryl sulfate, Tween 80, Solutol® HS15, propylene carbonate, and so forth. Both dispersion and solution embodiments are disclosed.
(g) Desai et al., WO 2006/034147 A2 (2006), discloses the use of dimethylsorbide as a solvent for formulating poorly water-soluble drugs such as ansamycins.
(h) Licari et al., U.S. patent application Ser. No. 11/595,005, filed Nov. 8, 2006, discloses nanoparticulate formulations of 17-AAG and a preferred polymorph of 17-AAG for use in such formulations.

However, there still remains room for improvement in the area of 17-AAG pharmaceutical formulations, as the prior art formulations suffer from one type of limitation or another. The present invention provides for improved 17-AAG pharmaceutical formulations.

### BRIEF SUMMARY OF THE INVENTION

In one aspect, there is provided a pharmaceutical formulation comprising (a) 17-AAG; (b) an ester of d-α-tocopheryl succinate and polyethylene glycol; and (c) a pharmaceutically acceptable, water-miscible organic solvent.

In another aspect, there is provided a method for preparing a 17-AAG pharmaceutical formulation ready for administration to a subject, comprising the steps of:
(i) providing a pharmaceutical formulation comprising (a) 17-AAG; (b) an ester of d-α-tocopheryl succinate and polyethylene glycol; and (c) a pharmaceutically acceptable, water-miscible organic solvent; and
(ii) diluting the pharmaceutical formulation with a pharmaceutically acceptable aqueous medium to produce a diluted pharmaceutical formulation ready for administration to a subject.

In another aspect, there is provided a method for administering 17-AAG to a subject, comprising the steps of:
(i) providing a pharmaceutical formulation comprising (a) 17-AAG; (b) an ester of d-α-tocopheryl succinate and polyethylene glycol; and (c) a pharmaceutically acceptable, water-miscible organic solvent;
(ii) diluting the pharmaceutical formulation with a pharmaceutically acceptable aqueous medium to produce a diluted pharmaceutical formulation; and
(iii) administering the diluted pharmaceutical formulation to a subject.

In another aspect, there is provided a method for preparing a 17-AAG pharmaceutical formulation ready for administration to a subject, comprising the steps of:
(i) providing a mixture of 17-AAG and an ester of d-α-tocopheryl succinate and polyethylene glycol;
(ii) dissolving the mixture of 17-AAG and an ester of d-α-tocopheryl succinate and polyethylene glycol in a pharmaceutically acceptable, water-miscible organic solvent to produce a pharmaceutical formulation; and
(iii) diluting the pharmaceutical formulation with a pharmaceutically acceptable aqueous medium to produce a diluted pharmaceutical formulation ready for administration to a subject.

In another aspect, there is provided a method for administering 17-AAG to a subject, comprising the steps of:
(i) providing a mixture of 17-AAG and an ester of d-α-tocopheryl succinate and polyethylene glycol;
(ii) dissolving the mixture of 17-AAG and an ester of d-α-tocopheryl succinate and polyethylene glycol in a pharmaceutically acceptable, water-miscible organic solvent to produce a pharmaceutical formulation;
(iii) diluting the pharmaceutical formulation with a pharmaceutically acceptable aqueous medium to produce a diluted pharmaceutical formulation; and
(iv) administering the diluted pharmaceutical formulation to a subject.

In another aspect, there is provided a kit comprising 17-AAG, an ester of d-α-tocopheryl succinate and polyethylene glycol, and a pharmaceutically acceptable, water-miscible organic solvent.

### DETAILED DESCRIPTION OF THE INVENTION

For conciseness, the following synonymous shortened terminology will be used hereinbelow: “tocopheryl ester” for “ester of d-α-tocopheryl succinate and polyethylene glycol”; “organic solvent” for “pharmaceutically acceptable, water-miscible organic solvent”; and “aqueous medium” for “pharmaceutically acceptable aqueous medium”.

The formulations of this invention are advantageous in various respects. They enable the preparation of formulations having higher final concentrations of 17-AAG (between about 2.5 and about 10 mg/mL, preferably about 2.5 to 5.0 mg/mL, when ready for administration), meaning a smaller volume of formulation needs to be administered for a subject to receive a given amount of 17-AAG. Where the subject receives the formulation by infusion, a smaller volume means a desirably shorter administration time. We have found that the formulations of our invention have enhanced storage stability when stored either at room temperature or under refrigeration. As shown by data provided hereinbelow, they the 17-AAG titer remains high for prolonged periods of time. Unlike prior art formulations containing Cremophor™, a commonly used formulation aid, the present formulations do not contain ingredients prone to causing allergic reactions.

Preferably, the tocopheryl ester is present in the formulation in an amount of from about 4 to about 10 parts by weight for each part by weight of 17-AAG in the formulation, more preferably in an amount of from about 2 to about 4 parts by weight for each part by weight of 17-AAG in the formulation.

The tocopheryl ester has a structure represented by formula (I)

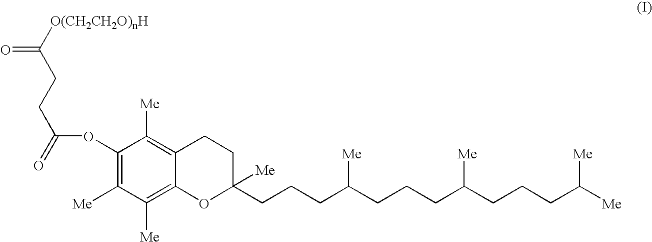

where the subscript n is an integer between 4 and 40, preferably between 8 and 32, more preferably between 18 and 24, and most preferably between 20 and 24. In another aspect, the polyethylene glycol preferably has an average molecular weight of between about 850 daltons and about 1600 daltons, more preferably an average molecular weight of between about 950 daltons and about 1050 daltons.

A specific preferred tocopheryl ester is one where polyethylene glycol moiety is polyethylene glycol 1000 (the numeral 1000 designating the approximate average molecular weight of the polyethylene glycol), which ester is available under the tradename Vitamin E TPGS™ from Eastman Chemical Company. Additional information on Vitamin E TPGS™ is available in the brochures Publication PCI 102-A, “Eastman Vitamin E TPGS NF: Properties and Applications” (June 2003), and Publication V-4D, “Eastman Vitamin E TPGS (d-Alpha-Tocopheryl Polyethylene Glycol-1000-Succinate” (November 2002), both by Eastman Chemical Company; the disclosures of which are incorporated herein by reference.

The organic solvent preferably is present in an amount of at least 4, more preferably at least 12, parts by weight for each part by weight of 17-AAG in the formulation. A preferred range for the organic solvent is between about 4 and about 30 parts by weight for each part by weight of 17-AAG in the formulation, more preferably between about 10 and about 20 parts by weight for each part by weight of 17-AAG in the formulation.

Examples of suitable organic solvents include ethanol, N,N-dimethylacetamide (DMA), N-methyl-2-pyrrolidone (NMP), dimethylsulfoxide (DMSO), and combinations thereof. (By “water-miscible”, it is meant that the solvent is infinitely miscible with water, that is, it is miscible in all proportions.) The organic solvent preferably is aprotic, a subclass that includes DMA, NMP, DMSO and combinations thereof. The pharmaceutically acceptable, water-miscible, aprotic organic solvent more preferably is DMA or NMP or combinations thereof.

In the preparation of a formulation of this invention, the 17-AAG and tocopheryl ester can be separately dissolved in respective first and second organic solvents (which can be the same or different), with the two solutions then combined. Alternatively, the 17-AAG and tocopheryl ester can be directly mixed together with the organic solvent, without making separate initial solutions.

For administration to a subject (which preferably is a mammal, more preferably a human), the formulation is diluted with a pharmaceutically acceptable aqueous medium, for example water for injection (WFI, especially sterile water for injection, or SWFI), 5% dextrose in WFI (D5W), saline, buffer, and the like, with the first two being preferred. Thus, in another aspect, a formulation of this invention further comprises a pharmaceutically acceptable aqueous medium.

The formulations of this invention can be stored either at room temperature or under refrigeration. Optionally, the formulations can be stored under an anaerobic atmosphere (e.g., under nitrogen or an inert gas) and/or protected from light.

We have found that a composition comprising the 17-AAG, the tocopheryl ester, and the organic solvent all together, though exhibiting good stability, nevertheless deteriorates over time, even when stored at temperatures as low as −20° C., with a decrease in 17-AAG assay of about 1% per week. Thus, in a preferred embodiment, the organic solvent is kept apart from the 17-AAG until shortly before administration. Such separation can be achieved through a kit comprising the 17-AAG, the tocopheryl ester, and the organic solvent. The kit comprises at least two containers, in which the 17-AAG and the organic solvent are in separate containers. Optionally, the kit further comprises the aqueous medium.

In one embodiment, the kit comprises a first container containing 17-AAG and the tocopheryl ester and a second container containing the organic solvent. In another embodiment, the 17-AAG, the tocopheryl ester, and the organic solvent are contained in respective first, second, and third containers. In another embodiment, the kit comprises a first container containing the 17-AAG and a second container containing the tocopheryl ester and the organic solvent. Yet other variations relating to the number of containers and the contents thereof are permissible.

Preferably, the amount of 17-AAG in the kit is a unit dose of 17-AAG. Where the kit comprises a container containing both the 17-AAG and the tocopheryl ester, the 17-AAG and the tocopheryl ester, either separately or jointly, are dissolved in a suitable solvent such as ethanol, methanol, dichloromethane, or acetonitrile, sterile filtering through a 0.22 micron filter into a sterile first container, and vacuum drying to remove substantially all the organic solvent, leaving behind a mixture of the 17-AAG and the tocopheryl ester. (The term “mixture” is used expansively to include a solid solution of the two components). Shortly before administration to a subject, the organic solvent is combined with the mixture to produce the three-component pharmaceutical formulation. This pharmaceutical formulation is then diluted with an aqueous medium, to produce a diluted pharmaceutical formulation ready for administration to a subject. The pharmaceutical formulation can be added to the aqueous medium or vice-versa, and mixed by vortexing or other conventional technique. The aqueous medium can be supplied as a further component of the kit or can be supplied by the care provider administering the 17-AAG. Preferably, the kit further comprises instructions for preparing, a pharmaceutical formulation comprising 17-AAG ready for administration to a subject, using the 17-AAG, tocopheryl ester, and the organic solvent in the kit. Instructions for the administration of the 17-AAG formulation also can be included.

The diluted pharmaceutical formulation is administered to a subject by an appropriate method, such as parenterally (especially intravenously) or orally. Preferably, the diluted pharmaceutical formulations of this invention are formulations for intravenous administration.

Geldanamycin is a well-known natural product, obtainable by culturing the producing organism, Streptomyces hygroscopicus var. geldanus NRRL 3602. 17-AAG is made semi-synthetically from geldanamycin, by reaction of geldanamycin with allylamine, as described in Sasaki et al., U.S. Pat. No. 4,261,989 (1981), the disclosure of which is incorporated herein by reference. Both geldanamycin and 17-AAG also are available commercially.

17-AAG can be used to treat a variety of proliferative disorders, such as, but not limited to, hyperproliferative diseases, including: cancers of the head and neck which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; treat ovarian cancer; small cell and non-small cell lung cancer; breast cancer sarcomas, such as fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma; neoplasms of the central nervous systems, particularly brain cancer; lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma. More particularly, cancers that can be targeted for treatment by 17-AAG include breast cancer, multiple myeloma, melanoma, colon cancer, lung cancer (especially non-small cell lung cancer (NSCLC)), prostate cancer, thyroid cancer, ovarian cancer, lymphoma, pancreatic cancer, and leukemia (especially chronic myelogenous leukemia (CML) and chronic lymphocytic leukemia or (CLL)).

Non-cancer disorders that are characterized by cellular hyperproliferation can also be treated by 17-AAG administered in accordance with this invention. Illustrative examples of such disorders include but are not limited to: atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis. irritant dermatitis, allergic dermatitis), periodontal disease (i.e., periodontitis), polycystic kidney disease, and type I diabetes. Other examples include vasculitis (e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis); gastrointestinal tract diseases (e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including ideopathic); and eustachean tube diseases (e.g., strictures of all causes including ideopathic).

17-AAG can be administered in combination with another active pharmaceutical ingredient (API), such as other anti-cancer or cytotoxic agents including alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, microtubule stabilizers, nucleoside (purine or pyrimidine) analogs, nuclear export inhibitors, proteasome inhibitors, topoisomerase (I or II) inhibitors, tyrosine kinase inhibitors. Specific anti-cancer or cytotoxic agents include β-lapachone, ansamitocin P3, auristatin, bicalutamide, bleomycin, bortezomib, busulfan, callistatin A, camptothecin, capecitabine, CC-1065, cisplatin, cryptophycins, daunorubicin, disorazole, docetaxel, doxorubicin, duocarmycin, dynemycin A, epothilones, etoposide, floxuridine, floxuridine, fludarabine, fluoruracil, gefitinib, geldanamycin, 17-DMAG, gemcitabine, hydroxyurea, imatinib, interferons, interleukins, irinotecan, maytansine, methotrexate, mitomycin C, oxaliplatin, paclitaxel, suberoylanilide hydroxamic acid (SAHA), thiotepa, topotecan, trichostatin A, vinblastine, vincristine, and vindesine. Preferred combinations are with gefitinib (Iressa™), bortezomib (Velcade), paclitaxel (Taxol™), docetaxel, thalidomide (Thalomid™), lenalidomide (Revlimid™), and Herceptin™.

Where a course of treatment entails a combination treatment involving 17-AAG and another API, such other API can be administered separately, in its own formulation, or, where amenable, can be administered as an additional component added to a formulation of this invention.

Using a pharmaceutical solution formulation of this invention, 17-AAG may be administered in a dose ranging from about 4 mg/m² to about 4000 mg/m², depending on the frequency of administration. A preferred dosage regimen for 17-AAG is about 450 mg/m² weekly (Banerji et al., Proc. Am. Soc. Clin. Oncol., 22, 199 (2003, abstract 797), “A Pharmacokinetically (PK)-pharmacodynamically (PD) Guided Phase I Trial of the Heat Shock Protein 90 (HSP90) Inhibitor 17-Allyl-17-demethoxygeldanamycin (17AAG)”). Alternatively, a dose of about 308 mg/m² weekly can be administered. See Goetz et al., Eur. J. Cancer, 38 (Supp. 7), S54-S55 (2002), “A phase I trial of 17-Allyl-Amino-Geldanamycin (17-AAG) in patients with advanced cancer.” Another dosage regimen is twice weekly, with doses ranging from 220 mg/m² to 340 mg/m² (preferably either 220 mg/m² or 340 mg/m²). A dosage regimen that can be used for combination treatments with another drug, such as docetaxel, is to administer the two drugs every three weeks, with the dose of 17-AAG being up to 650 mg/m² at each administration.

The practice of this invention can be further understood by reference to the following examples, which are provided by way of illustration and not of limitation.

### EXAMPLE 1

A stock solution of 17-AAG in DMA at a concentration of 100 mg/mL was prepared. An aliquot (0.125 mL) of the stock solution was transferred to a clean container (e.g., an Eppendorf tube). To this was added an aliquout (0.1 mL) of a stock solution of Vitamin E TPGS in DMA (50% w/v). The two solutions were mixed thoroughly. Then, water for injection (4.8 mL) was added, followed again by thorough mixing, to give a pharmaceutical formulation containing about 0.25 weight % 17-AAG, about 1.00 weight % vitamin E TPGS, about 3.23 weight % DMA, and about 95.52 weight % WFI.

The solution so prepared was stable (i.e., the 17-AAG remained dissolved) for more than 24 hr at room temperature.

### EXAMPLE 2

A series of 17-AAG formulations according to this invention were prepared, as tabulated in Table 1.

TABLE 1

| | | | Vitamin E | |
|---|---|---|---|---|
| | 17-AAG | DMA | TPGS | Ethanol |
| Example | (mg/mL) | (wt %) | (wt %) | (wt %) |
| A | 2.5 | 3 | 1 | 0 |
| B | 2.5 | 3.5 | 1 | 0 |
| C | 2.5 | 3 | 1 | 3 |
| D | 2.5 | 3 | 1 | 5 |
| E | 2.5 | 3 | 1 | 7 |
| F | 2.5 | 2 | 1 | 5 |
| G | 5 | 6 | 2 | 0 |
| H | 5 | 7 | 2 | 0 |
| I | 5 | 8 | 2 | 0 |
| J | 5 | 7 | 2.5 | 0 |
| K | 5 | 7 | 3 | 0 |
| L | 5 | 7 | 3.5 | 0 |

The stability of each of the examples at about 21° C. was evaluated as follows: after a certain amount of time had elapsed, an aliquot was filtered (0.22 μm filter) or centrifuged (13,500 rpm for 5 min) to remove any precipitated 17-AAG, diluted in methanol, and then assayed for 17-AAG content by high-pressure liquid chromatography. The results are tabulated in Tables 2 and 3.

TABLE 2

| | | Assayed 17-AAG Content (mg/mL) | | | |
|---|---|---|---|---|---|
| | | after Elapsed Time^{a} | | | |
| Example | Treatment | 0 hr | 22 hr | 48 hr | 192 hr |
| A | Centrifugation | 2.54 | 2.60 | 2.52 | 2.48 |
| A | Filtration | 2.53 | 2.47 | 2.40 | 2.33 |
| B | Centrifugation | 2.58 | 2.61 | 2.55 | 2.51 |
| B | Filtration | 2.55 | 2.51 | 2.41 | 2.36 |
| C | Centrifugation | 2.62 | 2.49 | 2.44 | 0.61 |
| C | Filtration | 2.59 | 2.43 | 2.32 | 0.56 |
| D | Centrifugation | 2.59 | 2.48 | 2.41 | 0.51 |
| D | Filtration | 2.58 | 2.35 | 2.26 | 0.48 |
| E | Centrifugation | 2.56 | 2.50 | 2.43 | 0.50 |
| E | Filtration | 2.57 | 2.40 | 2.28 | 0.46 |
| F^{b} | Centrifugation | 2.15 | 2.21 | 1.79 | 0.44 |
| F^{b} | Filtration | 2.15 | 2.09 | 1.69 | 0.40 |

^{a}Calculated original 17-AAG content = 2.5 mg/mL
^{b}Precipitation observed at 0 hr elapsed time

TABLE 3

| | | Assayed 17-AAG Content (mg/mL) | | | |
|---|---|---|---|---|---|
| | | after Elapsed Time^{a} | | | |
| Example | Treatment | 0 hr | 26 hr | 48 hr | 94 hr |
| G | Centrifugation | 4.96 | 4.66 | 4.88 | 4.85 |
| G | Filtration | 4.81 | 4.69 | 4.69 | 4.67 |
| H | Centrifugation | 4.94 | 4.76 | 4.85 | 4.90 |
| H | Filtration | 4.86 | 4.71 | 4.65 | 4.64 |
| I | Centrifugation | 5.00 | 4.74 | 4.88 | 4.89 |
| I | Filtration | 4.77 | 4.76 | 4.68 | 4.69 |
| J | Centrifugation | 4.87 | 4.64 | 4.79 | 4.78 |
| J | Filtration | 4.74 | 4.65 | 4.65 | 4.68 |
| K | Centrifugation | 4.76 | 4.63 | 4.66 | 4.67 |
| K | Filtration | 4.72 | 4.61 | 4.55 | 4.59 |
| L | Centrifugation | 4.84 | 4.54 | 4.68 | 4.74 |
| L | Filtration | 4.71 | 4.59 | 4.57 | 4.58 |

^{a}Calculated original 17-AAG content = 5.0 mg/mL

The data show that formulations A-L (especially the ethanol-free ones) are stable for at least 48 hrs, and in many instances, for up to 192 hrs, as evidenced by the continued high 17-AAG content, as determined by HPLC peak areas.

### EXAMPLE 3

Additional formulations using various combinations of pharmaceutically acceptable, water-miscible organic solvents were prepared. Their compositions are given in Table 4.

TABLE 4

| | 17-AAG | Vitamin E TPGS | 1st Solvent | 2nd Solvent |
|---|---|---|---|---|
| Example | (mg/mL) | (wt %) | (wt %) | (wt %) |
| M | 2.5 | 1 | DMA (1%) | NMP (3%) |
| N | 2.5 | 1 | Ethanol (1%) | NMP (3%) |
| O | 2.5 | 1 | DMA (3%) | Ethanol (1%) |
| P | 5.0 | 1 | DMA (6%) | Ethanol (2%) |

Visual observations of stability are recorded in Table 5.

TABLE 5

| | |
|---|---|
| Time | Visual Appearance (Example) |

| (hrs) | M | N | O | P |
|---|---|---|---|---|
| 0.0 | C | C | C | C |
| 1.8 | C | C | C | FMP |
| 4.2 | C | C | C | FMO/SP |
| 16.6 | C | FMP | C/FMP | LMP/PPT |
| 27.7 | C | LMP | LMP | LMP/PPT |
| 30.7 | C | LMP | LMP | — |
| 41.6 | C | LMP | LMP | — |
| 51.0 | C | LMP | LMP | — |
| 71.3 | C | LMP | LMP | — |

Key:
C = clear, intense purple color
FMP = faintly milky purple
LMP = little milky purple (may contain slight precipitate)
SP = contains slight precipitate
PPT = precipitate

The above results again evidence the stability of formulations of this invention. As before, better results are obtained with the formulations that contain only an aprotic solvent as the pharmaceutically acceptable, water-miscible organic solvent, that is, are ethanol-free.

### EXAMPLE 4

This example illustrates the kit approach to the preparation of four formulations (Examples Q through T) of this invention, each having the following final constitution:

| | | |
|---|---|---|
| 17-AAG | 5 | mg/mL |
| Vitamin E TPGS ™ | 2 | wt % |
| DMA | 3.33 | wt % |
| NMP | 3.33 | wt % |

| | |
|---|---|
| SWFI | qs |

Referring to Table 6, the indicated amounts of 17-AAG were weighed out in a flask. The indicated amounts of Vitamin E TPGS™ stock solution were added to the 17-AAG, to form a slurry or sludge-like mixture. Solvent (methanol, ethanol, acetonitrile, or dichloromethane, as noted) was added in the indicated amounts to fully dissolve the 17-AAG and Vitamin E TPGS™. It was observed that the effectiveness of the solvents fell in the following order: methanol˜dichloromethane>ethanol>acetonitrile. The methanol, ethanol, acetonitrile, or dichloromethane, as the case may be, was then removed by rotary evaporation and vacuum-dried overnight, leaving a waxy, film-like residue of 17-AAG and Vitamin E TPGS™, corresponding to the arrangement in a kit in which the 17-AAG and Vitamin E TPGS™ are jointly contained in a single container. Next, a 1:1 v:v mixture of DMA and NMP was added in the indicated amounts to dissolve the 17-AAG and Vitamin E TPGS™. Lastly, sterile water for injection was added in the indicated amounts to prepare the finalized formulations.

TABLE 6

| | Example | Example | | |
|---|---|---|---|---|
| Material | Q | R | Example S | Example T |
| 17-AAG (mg) | 24.2 | 24.2 | 27.2 | 27.7 |
| Vitamin E TPGS ™ | 50% (w/v) | 50% (w/v) | 50% (w/v) in | 50% (w/v) in |
| stock solution | in MeOH | in EtOH | acetonitrile | CH₂Cl₂ |
| Vitamin E TPGS ™ | 0.194 | 0.194 | 0.218 | 0.222 |
| stock solution (mL) | | | | |
| Solvent (mL) | MeOH | EtOH | Acetonitrile | CH₂Cl₂ (~1) |
| | (~1) | (~5) | (~>15) | |
| 1:1 v:v DMA/NMP | 0.323 | 0.323 | 0.363 | 0.369 |
| (mL) | | | | |
| Sterile WFI (mL) | 4.5 | 4.5 | 5.1 | 5.2 |
| Final volume (mL) | 4.84 | 4.84 | 5.44 | 5.54 |

The stability of the formulations of Examples Q through T was monitored, with results as noted below in Table 7.

TABLE 7

| Time after | | | | |
|---|---|---|---|---|
| preparation (hrs) | Example Q | Example R | Example S | Example T |
| 0.0 | C | C | C | C (?)/FMP |
| 1.5 | C | C | C | FMP |
| 2.5 | C | C | C | SMP |
| 6.0 | C | C | C | MP |
| 9.5 | C?/FMP | C | C | MP |
| 12.0 | FMP | C | C | MP |
| 24.0 | SMP | C | C | MP |
| 37.0 | MP | C | C | MP |
| 42.7 | MP | C | C | MP |

Key:
C = clear, intense purple color
FMP = faintly milky purple
LMP = little milky purple (may contain slight precipitate)
SP = contains slight precipitate
PPT = precipitate

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

We claim:

1. A pharmaceutical formulation comprising (a) 17-allylamino-17-demethoxy-geldanamycin (“17-AAG”); (b) an ester of d-α-tocopheryl succinate and polyethylene glycol; and (c) a pharmaceutically acceptable, water-miscible organic solvent.

2. A formulation according to claim 1, wherein the ester of d-α-tocopheryl succinate and polyethylene glycol has a structure represented by formula (I) 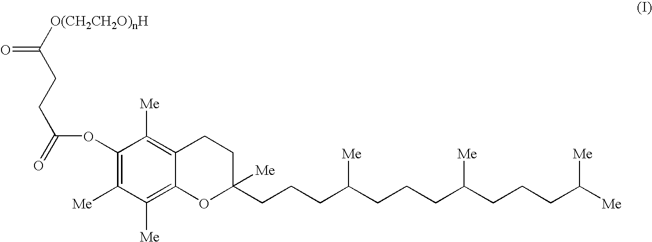
where the subscript n is an integer between 4 and 40.

3. A formulation according to claim 1, wherein, in the ester of d-α-tocopheryl succinate and polyethylene glycol, the polyethylene glycol is polyethylene glycol 1000.

4. A formulation according to claim 1, wherein the ester of d-α-tocopheryl succinate and polyethylene glycol is present in an amount of from about 4 to about 10 parts by weight for each part by weight of 17-AAG in the formulation.

5. A formulation according to claim 1, wherein the pharmaceutically acceptable, water-miscible organic solvent is aprotic.

6. A formulation according to claim 1, wherein the pharmaceutically acceptable, water-miscible organic solvent is N,N-dimethylacetamide, N-methyl-2-pyrrolidone, dimethylsulfoxide, or combinations thereof.

7. A formulation according to claim 1, wherein the pharmaceutically acceptable, water-miscible organic solvent is present in an amount of at least 4 parts by weight for each part by weight of 17-AAG in the formulation.

8. A formulation according to claim 1, further comprising a pharmaceutically acceptable aqueous medium.

9. A formulation according to claim 8, wherein the pharmaceutically acceptable aqueous medium is water for injection or 5% dextrose in water for injection.

10. A method for preparing a 17-allylamino-17-demethoxygeldanamycin (“17-AAG”) pharmaceutical formulation ready for administration to a subject, comprising the steps of:
(i) providing a pharmaceutical formulation comprising (a) 17-AAG; (b) an ester of d-α-tocopheryl succinate and polyethylene glycol; and (c) a pharmaceutically acceptable, water-miscible organic solvent; and
(ii) diluting the pharmaceutical formulation with a pharmaceutically acceptable aqueous medium to produce a diluted pharmaceutical formulation ready for administration to a subject.

11. A method for administering 17-allylamino-17-demethoxygeldanamycin (“17-AAG”) to a subject, comprising the steps of:
(i) providing a pharmaceutical formulation comprising (a) 17-AAG; (b) an ester of d-α-tocopheryl succinate and polyethylene glycol; and (c) a pharmaceutically acceptable, water-miscible organic solvent;
(ii) diluting the pharmaceutical formulation with a pharmaceutically acceptable aqueous medium to produce a diluted pharmaceutical formulation; and
(iii) administering the diluted pharmaceutical formulation to a subject.

12. A method for preparing a 17-allylamino-17-demethoxygeldanamycin (“17-AAG”) pharmaceutical formulation ready for administration to a subject, comprising the steps of:
(i) providing a mixture of 17-AAG and an ester of d-α-tocopheryl succinate and polyethylene glycol;
(ii) dissolving the mixture of 17-AAG and an ester of d-α-tocopheryl succinate and polyethylene glycol in a pharmaceutically acceptable, water-miscible organic solvent to produce a pharmaceutical formulation; and
(iii) diluting the pharmaceutical formulation with a pharmaceutically acceptable aqueous medium to produce a diluted pharmaceutical formulation ready for administration to a subject.

13. A method for administering 17-allylamino-17-demethoxygeldanamycin (“17-AAG”) to a subject, comprising the steps of:
(i) providing a mixture of 17-AAG and an ester of d-α-tocopheryl succinate and polyethylene glycol;
(ii) dissolving the mixture of 17-AAG and an ester of d-α-tocopheryl succinate and polyethylene glycol in a pharmaceutically acceptable, water-miscible organic solvent to produce a pharmaceutical formulation;
(iii) diluting the pharmaceutical formulation with a pharmaceutically acceptable aqueous medium to produce a diluted pharmaceutical formulation; and
(iv) administering the diluted pharmaceutical formulation to a subject.

14. A method according to claim 13, wherein the ester of d-α-tocopheryl succinate and polyethylene glycol has a structure represented by formula (I) 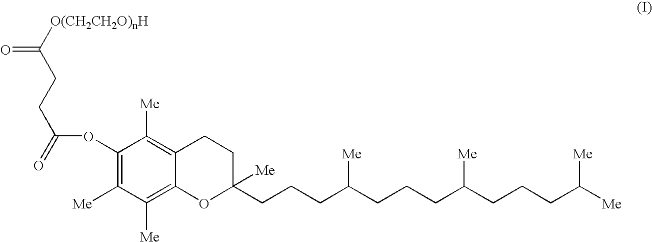
where the subscript n is an integer between 4 and 40.

15. A method according to claim 14, wherein the polyethylene glycol in the ester of d-α-tocopheryl succinate and polyethylene glycol is polyethylene glycol 1000.

16. A method according to claim 15, wherein the ester of d-α-tocopheryl succinate and polyethylene glycol is present in an amount of from about 4 to about 10 parts by weight for each part by weight of 17-AAG.

17. A method according to claim 13, wherein the pharmaceutically acceptable, water-miscible organic solvent is N,N-dimethylacetamide, N-methyl-2-pyrrolidone, dimethylsulfoxide, or combinations thereof.

18. A method according to claim 17, wherein the pharmaceutically acceptable, water-miscible organic solvent is present in an amount of at least 4 parts by weight for each part by weight of 17-AAG.

19. A method according to claim 13, wherein, in the diluted pharmaceutical formulation, the 17-AAG is present at a concentration of between about 2.5 and about 10 mg/mL.

20. A method according to claim 13, wherein the pharmaceutically acceptable aqueous medium is water for injection or 5% dextrose in water for injection.

21. A kit comprising 17-allylamino-17-demethoxygeldanamycin (“17-AAG”), an ester of d-α-tocopheryl succinate and polyethylene glycol, and a pharmaceutically acceptable, water-miscible organic solvent.

22. A kit according to claim 21, wherein the 17-AAG and the ester of d-α-tocopheryl succinate and polyethylene glycol are contained in a first container and the pharmaceutically acceptable, water-miscible organic solvent is contained in a second container.

23. A kit according to claim 21, wherein the 17-AAG, the ester of d-α-tocopheryl succinate and polyethylene glycol, and the pharmaceutically acceptable, water-miscible organic solvent are contained in respective first, second and third containers.

24. A kit according to claim 21, further comprising a pharmaceutically acceptable aqueous medium.

25. A kit according to claim 21, further comprising instructions for preparing a pharmaceutical formulation comprising 17-AAG ready for administration to a subject using the 17-AAG, the ester of d-α-tocopheryl succinate and polyethylene glycol, and the pharmaceutically acceptable, water-miscible organic solvent in the kit.

26. A kit according to claim 21, wherein the amount of 17-AAG in the kit is a unit dose of 17-AAG.
